(19) United States
(12) Patent Application Publication
(10) Pub. No.: US 2020/0360037 A1
(43) Pub. Date: Nov. 19, 2020

(54) FORCEPS AND KITS FOR FORMING THE SAME

(71) Applicant: DTR Medical Limited, Swansea (GB)

(72) Inventors: Richard HALL, Harrogate (GB); Tim FLOOD, Hartburn (GB); Matthew FRANKS, Bristol (GB)

(21) Appl. No.: 16/441,520

(22) Filed: Jun. 14, 2019

(30) Foreign Application Priority Data
May 17, 2019 (GB) 1906977.2

(51) Int. Cl. A61B 17/29

(52) U.S. Cl. CPC A61B 17/2909; A61B 2017/2912; A61B 2017/2927; A61B 2017/2939

(57) ABSTRACT

The present invention provides forceps (1, 101) for use in ear, nose and throat procedures. The forceps comprise: a forward handle (2, 102) and a rear handle (3); at least one movable jaw (5) proximate to, or at, a front of the forceps, said jaw being coupled to the rear handle such that movement of the rear handle causes movement of the at least one jaw between a first operating position and a second operating position; the forward handle comprising a forward-facing digit contact surface (11) and a rearward-facing digit contact surface (12). A kit for forming forceps is also provided, as is a method of making forceps.

### CROSS-REFERENCE TO RELATED APPLICATIONS

This application claims priority to, and the benefit of, United Kingdom (GB) Patent Application No. 1906977.2 filed with the Intellectual Property Office (IPO) of the United Kingdom on May 17, 2019, which his hereby incorporated by reference.

### BACKGROUND OF THE INVENTION

The present disclosure relates to forceps, particularly, but not exclusively those suitable for use in ear, nose and throat (“ENT”) procedures.

The present invention concerns forceps. More particularly, but not exclusively, this invention concerns forceps for use in ear, nose and throat procedures, kits for forming the same, and methods for making forceps using such kits.

Conventional ENT forceps, such as crocodile forceps, have one or more (and typically two) jaws which co-operate to grasp or cut. The jaws are typically located at the end of an elongate shaft which, in use, may be inserted (at least partially) into a body cavity, such as an ear or nose. One or both of the jaws may be movable, such movement being caused by movement of one of the forceps' two handles. The forceps have a forward handle and a rear handle, the forward handle comprising a finger loop for the insertion of one or more of the user's fingers and the rear handle comprising a thumb loop for the insertion of a user's thumb. Typically, the movable jaw(s) is coupled to the forward handle such that movement of the forward handle causes movement of the movable jaw(s). Unskilled users of these forceps often find it difficult to keep the jaw end of the forceps still, the forceps having a natural tendency to “waggle” up and down as the user operates the forceps. This is obviously undesirable, given the intended use of the forceps.

The jaws provided with conventional ENT forceps may take many different forms. For example, the jaws may be purely for grasping, as in conventional alligator or crocodile forceps. The jaws may be for cutting, such as in a Hartmann nasal cutting forceps. Forceps are typically constructed so that one type of jaw is used with one type of forceps. There is therefore a desire to provide forceps which may be more cheaply made and with fewer different parts.

The present invention seeks to mitigate the above-mentioned problems. Alternatively or additionally, the present invention seeks to provide an improved forceps, such as those suitable for use in ear, nose and throat procedures.

### SUMMARY OF THE INVENTION

The present invention provides in accordance with a first aspect of the present invention, forceps for use in ear, nose and throat procedures, the forceps comprising:

a forward handle and a rear handle;
at least one movable jaw proximate to, or at, a front of the forceps, said jaw being coupled to the rear handle such that movement of the rear handle causes movement of the at least one jaw between a first operating position and a second operating position,
the forward handle comprising a forward-facing digit contact surface and a rearward-facing digit contact surface.

The forward-facing and rearward-facing digit contact surfaces facilitate stabilisation of the forceps during use. For the avoidance of doubt, “digit” refers to finger(s). The rear handle is typically moved between operating positions (typically a closed and open position, for example) by a user's thumb. The applicant has discovered that the end of the forceps provided with the jaw(s) has less of a tendency to move when the moveable jaw is coupled to the thumb-actuated handle, when the front handle which is usually held by the fingers of a user is provided with a forward-facing surface and a rearward-facing surface on which to contact a user's fingers.

The terms “forward”, “rear”, “front”, “rearward” and any associated terms used herein are in no way limiting but are merely used to indicate relative positions and/or orientations of certain features.

For the avoidance of doubt, “ear, nose and throat” indicates that the forceps are suitable for use in ear, nose and throat procedures (often referred to as “ENT” procedures). Those skilled in the art will realise that such forceps are typically designed and intended for a particular use, and may not be designed or intended for use in multiple different procedures that involve each of the ear, nose and throat.

The forceps may comprise more than one jaw. The forceps may be configured so that more than one jaw is coupled to the rear handle so that movement of the rear handle causes movement of said more than one jaws. In some cases, one (and only one) jaw may be coupled to the rear handle such that movement of the rear handle causes movement of said one (and only one) jaw.

If one, and only one jaw, is coupled with the rear handle to be movable, then the movable jaw may be an upper jaw or a lower jaw, and optionally an upper jaw.

The forceps may comprise a support which is rigidly coupled to the forward handle. The support may act as a shank. If the forceps comprises a non-movable jaw, the non-movable jaw is optionally coupled to the support.

The forceps may comprise a jaw member comprising a jaw and an interface portion comprising a male or female portion for mating with a corresponding female or male portion provided by the support to secure the jaw member (and therefore the jaw) to the support.

Said jaw interface portion may be provided with a male part and the support may be provided with a corresponding female part.

The male and female parts optionally together form an interference-fit arrangement.

If the forceps comprises two jaws, then in the first operating position the at least one movable jaw may be further away from the other jaw than when the at least one movable jaw is in the second operating position.

According to a second aspect of the invention there is also provided forceps suitable for ear, nose and throat procedures, the forceps comprising:

a forward handle and a rear handle;
a movable first jaw proximate to, or at, a front of the device, said movable jaw being coupled to the forward or the rear handle such that movement of the respective forward or rear handle causes movement of the at least one jaw between a first operating position and a second operating position; and
a jaw member comprising a second jaw which co-operates with the first jaw and an interface portion comprising a male or female portion which is mated with a corresponding female or male portion provided by a jaw support to form an interference fit to secure the jaw member to the jaw support.

The applicant has discovered that the provision of an interference fit has provided a forceps which is simple to make and versatile in that different types of second jaws may be used in combination with the support.

In the first operating condition, the first jaw is optionally further from the second jaw than when the first jaw is in the second operating position.

The first jaw is optionally coupled to the rear handle such that movement of the rear handle causes movement of the first jaw between a first operating position and a second operating position. It has been discovered that this may provide an instrument which is easier to use and which has less of a tendency to “waggle” during use.

The support is typically rigidly coupled to the one of the forward and rear handles which is not coupled to the movable first jaw.

The forward handle may be provided with a forward-facing digit contact surface and a rearward-facing digit contact surface.

For the avoidance of doubt, the statements below pertain to the forceps of both the first and second aspects of the present invention. In the statements below, the term “jaws” may be taken, in relation to the forceps of the second aspect of the present invention to mean the first and second jaws. Likewise, reference to “movable jaw(s)” may be taken, in relation to the forceps of the second aspect of the present invention, to be the first jaw.

The forceps may comprise at least two and optionally two (and only two) jaws. The jaws may be configured for a particular task. For example, the jaws may be configured for grasping. In this case, one or both of the jaws may be provided with a textured surface. In this case, the first operating position may be an open position in which the jaws are open to receive an article to be grasped. The second operating position may be a position in which the jaws are closer together than in the first operating position. This does not mean that the jaws have to be closed when in the second operating position. Alternatively or additionally, the jaws may be configured to cut, in which case the first and second jaws may be configured to provide a cutting action when brought into proximity to one another.

The movable jaw(s) which is coupled to the respective handle may be coupled to the handle with a movable coupling member. The coupling member may optionally be elongate. The coupling member may optionally be pivotally attached to the respective handle. Pivotal attachment facilitates movement of the coupling member with movement of the handle. The coupling member may be coupled to the respective handle such that movement of the respective handle causes linear translational motion (i.e. straight line motion) of the coupling member. The coupling member may be attachable to the handle in a plurality of positions. This allows the separation of the front and rear handles to be set at one of a plurality of values. This is useful so that the forceps can be used by people having different hand sizes.

The movable coupling member is operable to move relative to the support (if present). The support may be elongate, for example. The movable coupling member may be disposed at least partially within the support. In this case, the support may be tubular, for example, with the movable coupling member being at least partially disposed in the tubular support.

The forceps may comprise a coupler to convert movement of the movable coupling member (for example, linear translational movement) into movement of the movable jaw(s). The movement of the movable jaw(s) may comprise a rotational movement.

The movable jaw(s) may be mounted for rotation about a pivot. The movement about the pivot is the movement between first operating and second operating positions. The pivot may be attached to the support, if present.

The coupler may comprise a pin and groove arrangement. One of the pin and groove may be associated with the movable jaw(s). The other of the pin and groove may be associated with the movable coupling member. The movable jaw(s) may be provided as a jaw member comprising a jaw portion and a jaw member coupler portion, the jaw member coupler portion comprising a pin or a groove. The jaw member coupler portion may also comprise an aperture for facilitating pivoting movement of the jaw member. The aperture may be configured to receive a member about which the jaw member may pivot.

One or both of the forward and rear handles may be provided with a digit loop for receipt of one or more operating digits.

The rearward facing digit contact surface (if present) may be below the forward facing digit contact surface (if present). One or both of the forward and rearward facing digit contact surfaces may be provided by a digit loop for the receipt of one or more operating digits. The forward-facing digit contact surface may be concave when viewed from the front of the forceps. The rearward-digit contact surface may be concave when viewed from the rear of the forceps.

In accordance with a third aspect of the present invention, there is provided a kit for assembly into forceps suitable for ear, nose and throat procedures, the kit comprising:

a jaw support; and
a plurality of sets of jaw members,
each set of jaw members comprising (i) a first jaw member comprising a first jaw and a jaw member coupler portion to facilitate movement of said jaw member when mounted for use and (ii) a second jaw member comprising a second jaw and a jaw interface portion comprising a male or female portion to facilitate mating with a corresponding female or male portion provided by the jaw support, thereby form an interference fit to secure the second jaw member to the jaw support,
the jaw member coupler portions of the first jaw members of the plurality of sets being substantially the same;
the jaw interface portions of the second jaw members of the plurality of sets being substantially the same; and
at least the first jaws and/or the second jaws of the plurality of sets being different.

The kit of the third aspect of the present invention facilitates the simple and rapid construction of forceps of different types by allowing the manufacturer to put different jaws in the same jaw support. For example, at least one of the sets of jaw members may comprise a first jaw comprising a gripping portion and a second jaw comprising a gripping portion. In use, the first and second jaws may be movable relative to one another between a position in which the first and second jaws are in a gripping configuration and a position in which the first and second jaws are in a release configuration. At least one of the sets of jaws may comprise a first jaw and a second jaw, at least one of which comprises a shearing portion. In use, the first and second jaws may be movable relative to one another between a shearing configuration and an open configuration. At least one of the sets of jaws may comprise a first jaw and a second jaw, at least one of which comprises a chopping portion. In use, the first and second jaws may be movable relative to one another between a chopping configuration and an open configuration.

The forceps support may comprise those features described above in relation to the forceps in accordance with the first and second aspects of the present invention. For example, the kit may comprise two handles which, in use, form the forward and rear handles of the forceps. The kit may comprise a coupling member configured to couple a movable handle to a movable jaw.

The kit may comprise a kit of unassembled parts or may comprise part-built forceps with components missing, those missing components comprising at least the first and second jaw members.

The kit is typically used by manufacturers, and would not typically be provided to consumers. In accordance with a fourth aspect of the present invention, there is therefore provided a method of manufacturing forceps in accordance with the second aspect of the present invention, the method comprising:

Providing a kit in accordance with the third aspect of the present invention;
Selecting a set of jaw members; and
Making forceps in accordance with the second aspect of the present invention using the selected jaw members.

The method may optionally comprise packaging said forceps, and optionally sterilising said forceps (optionally after packaging). The method may also optionally comprise delivering said forceps to a customer.

It will, of course, be appreciated that features described in relation to one aspect of the present invention may be incorporated into other aspects of the present invention. For example, the forceps of the first aspect of the present invention may incorporate any of the features described with reference to the forceps of the second aspect of the present invention and vice versa.

### DESCRIPTION OF THE DRAWINGS

Embodiments of the present invention will now be described by way of example only with reference to the accompanying schematic drawings of which:

FIG. 1 shows a side-on view of forceps according to a first embodiment of the invention;

FIG. 2 shows an exploded perspective view of part of the forceps shown in FIG. 1; and

FIG. 3 shows a cut-away side-on view of part of the forceps shown in FIG. 1, with the jaws in a closed configuration;

FIG. 4 shows a cut-away side-on view of part of the forceps shown in FIG. 1, with the jaws in an open configuration; and

FIG. 5 shows a side-on view of forceps according to a second embodiment of the invention.

### DETAILED DESCRIPTION

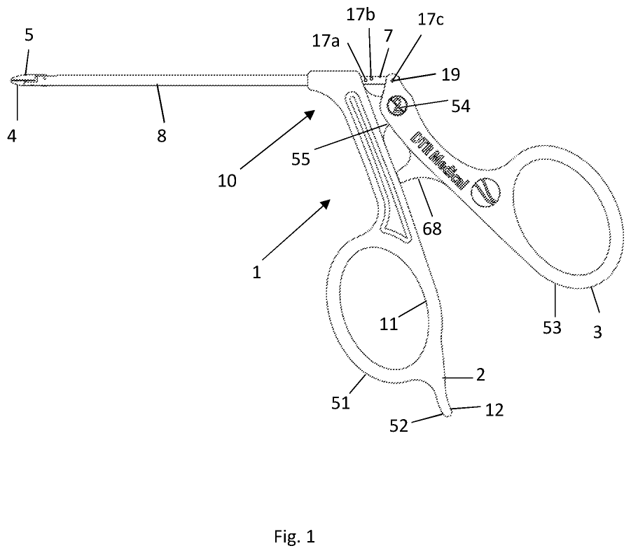

An example of an embodiment of forceps in accordance with the first and second aspects of the present invention is shown in FIGS. 1 to 4. Referring to FIG. 1, the forceps are denoted generally by reference number 1 and comprise a forward handle 2, a rear handle 3, a jaw 5 proximate to, or at, a front of the forceps 1, said jaw 5 being coupled to the rear handle 3 such that movement of the rear handle 3 causes movement of the jaw 5 between a first operating position and a second operating position. The forward handle 2 comprises a forward-facing digit contact surface 11 and a rearward-facing digit contact surface 12. The forward-facing digit contact surface 11 and the rearward-facing digit contact surface 12 enable the user to stabilise the forceps 1 during operation of the rear handle 3 by placing one or more fingers against the forward-facing digit contact surface 11 and one or more fingers against the rearward-facing digit contact surface 12. Forward-facing digit contact surface 11 is formed by part of a finger loop 51 in which one or more fingers may be placed, and the forward-facing digit-contact surface 11 is concave when viewed from the front of the forceps. Rearward-facing digit contact surface 12 is formed by arm 52 which projects from the finger loop 51. Rearward-facing digit contact surface 12 is concave when viewed from the rear of forceps 1. Handle 3 is provided with a thumb loop 53.

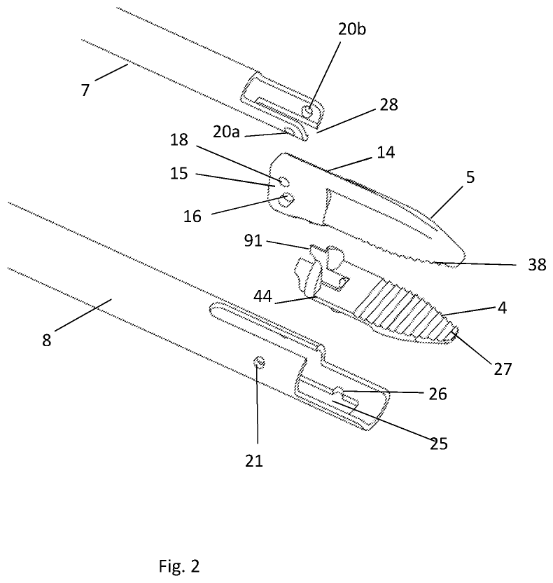

The construction and operation of forceps 1 will now be described with reference to FIGS. 1 to 4. The forceps 1 comprises a shank, denoted generally by the reference numeral 10, comprising forward handle 2 which is rigidly coupled to jaw support 8. Jaw support 8 is generally tubular in shape as can best be seen in FIGS. 2 to 4. Lower jaw 4 is rigidly coupled to jaw support 8 as is described in greater detail below.

Rear handle 3 is operably coupled to movable jaw 5 so that movement of rear handle 3 causes movement of movable jaw 5 between first and second operating positions. Rear handle 3 is pivotally attached at pivot 54 to a rearwardly-projecting protrusion 55 provided on forward handle 2. Rear handle 3 is pivotally attached at its end at pivot point 19 to movable coupling member 7. Movement of rear handle 3 causes linear translational movement of coupling member 7. Coupling member 7 is generally tubular in shape as can best be seen in FIGS. 2 to 4, and coupling member 7 is received within, and slides relative to, jaw support 8.

The operation of the forceps will now be described in more detail with reference to FIGS. 2 to 4. As mentioned above, jaw 4 is rigidly coupled to jaw support 8. In this connection, jaw 4 is provided as part of a jaw member 44 which provides, in addition to jaw 4, male formations 9 and 91 which facilitate securing of the jaw 4 to jaw support 8. Male formation 9 comprises an elongate portion and two laterally-projecting hemi-cylindrical portions which are received by corresponding elongate 25 and hemi-cylindrical 26 female portions provided in jaw support 8. The female and male portions are sized such that there is an interference fit between the two. In this connection, the female portions on support 8 are sized to be slightly smaller than the corresponding male portions on jaw member 44 to achieve the interference fit. Furthermore, rearwardly-projecting male portion 91 is received in a cavity formed by the wall of the jaw support 8.

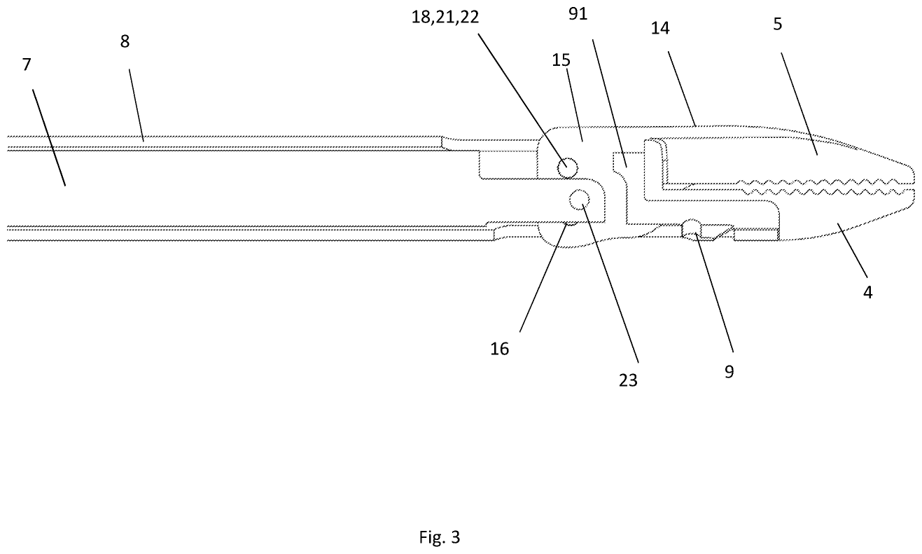

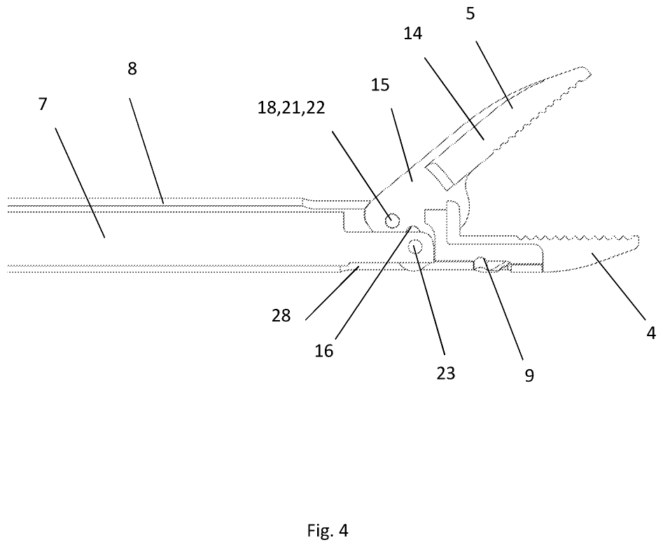

Movable jaw 5 is pivotally attached to jaw support 8 to facilitate pivotal movement of jaw 5 relative to jaw 4. Movable jaw member 14 comprises movable jaw 5 and jaw member coupler portion 15 which extends rearwardly from movable jaw 5. Jaw member coupler portion 15 is provided with an aperture 18 therethrough which receives a pin 22 which extends through aperture 18 and into apertures 21 provided in jaw support 8. This facilitates rotational movement of jaw member 14 and therefore jaw 5 relative to jaw support 8 and static jaw 4. Movement of movable jaw 5 is effected using a pin-and-groove arrangement. A groove 16 is provided in jaw member coupler portion 15 as can be seen in FIGS. 3 and 4. A pin 23 is attached to movable coupling member 7 at apertures 20a, 20b (see FIG. 2). Pin 23 is located in groove 16. If we consider FIG. 3, the jaws 4, 5 are in a closed configuration. As rear handle 3 is moved, movable coupling member 7 slides forwards, moving pin 23 forwards. The position of the pin 23 in groove 16 changes and causes rotation of movable jaw 5 from a closed configuration to an open configuration, as can be seen in FIG. 4. Movement of the rear handle in the opposite direction causes movement of the movable coupling member 7 backwards, which in turn returns the jaws 4, 5 to a closed configuration (see FIG. 3).

Both the movable coupling member 7 and jaw support 8 are provided with slots (labelled 28 in the case of movable coupling member 7) to accommodate the lower part of jaw member coupler portion 15.

The position on the movable coupling member 7 at which the end of the rear handle 3 is attached may be chosen from one of three positions, 17a, 17b, 17c to facilitate use by users of differing hand sizes. Alternatively, the forceps 1 may be provided with only one such position 17a, b, c.

Limiter 68, a forwardly-extending projection provided on rear handle 3, is provided to limit the motion of the forward and rear handles relative to one another.

Jaws 4, 5 are provided with a ridged surface 27, 38 to facilitate grasping of an object therebetween. The forceps 1 are crocodile or alligator forceps which are typically used to extract foreign bodies from cavities, such as the ear or the nose. The ridged surfaces 27, 38 facilitate grasping of those foreign bodies.

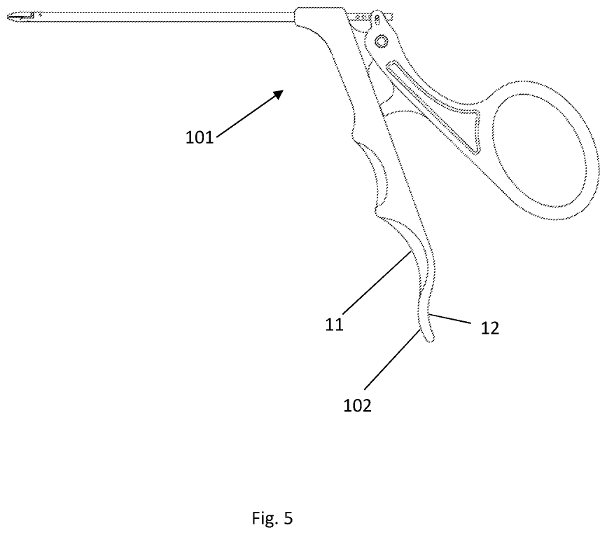

A second example of an embodiment of the present invention is shown in FIG. 5. The forceps are denoted generally by reference numeral 101 and are essentially the same as the forceps of FIGS. 1 to 4, except that forward handle 102 does not have a finger loop, merely forward-facing and rearward-facing digit contact surfaces.

A kit in accordance with an example of an embodiment of the present invention will now be described with reference to the forceps of FIGS. 1 to 5. The kit comprises all of the parts of the forceps 1, 101, without the jaws, and a plurality of sets of jaw members, each set of jaw members being different from the other. Each set of jaw members comprises a jaw member to be rigidly coupled to jaw support 8 and a jaw member which is, in use, movable relative to the jaw member rigidly coupled to jaw support 8. Each of the jaw members which is to be movable comprises common jaw member coupler portions to facilitate movement of the respective jaw member in the assembled forceps. Each of the jaw members which is to be rigidly coupled to the jaw support comprises common jaw interface portions to enable secure fitment to the jaw support. However, the jaws of different sets are different. One set of jaws may be the grasping jaws as described above in relation to the forceps of FIGS. 1 to 5. Another set of jaws may be a scissors-style set of jaws, while another set of jaws may be a sampling set of jaws, suitable for both cutting a tissue sample and retaining it in the jaws for examination.

An example of an embodiment of a method of manufacture of forceps in accordance with the second aspect of the present invention using a kit in accordance with the third aspect of the present invention will now be described. A kit is provided as described above. The manufacturer would then choose a set of jaw members (in one example, jaw members provided with grasping jaws). The jaw member to be rigidly coupled to the jaw support 8 would be attached to the jaw support and the corresponding jaw member which is to be movable would be coupled to the movable coupling member (the slidable rod). The assembled forceps would then be packaged (for example, inserted into a bag), sterilised (for example, by exposure to suitable electromagnetic radiation) and dispatched to a customer.

Whilst the present invention has been described and illustrated with reference to particular embodiments, it will be appreciated by those of ordinary skill in the art that the invention lends itself to many different variations not specifically illustrated herein. By way of example only, certain possible variations will now be described.

The examples above demonstrate the use of a tubular fixed member in which is disposed a moveable tubular member which is moveable to cause movement of the jaw. Those skilled in the art will realise that other arrangements are possible. For example, the moveable member may be disposed on top of the non-moveable member which is configured as a shank, the movable member being movable backwards and forwards along the non-movable member.

The examples above demonstrate forceps with a grasping jaw arrangement. Those skilled in the art will realise that other jaw arrangements are possible. For example, cutting jaw arrangements may be used.

The examples above demonstrate forceps with a jaw arrangement in which the lower jaw is fixed and the upper jaw is configured to move. Those skilled in the art will realise that the upper jaw may be fixed and the lower jaw is configured to move. Certain examples of forceps (those in accordance with the first aspect of the present invention) may be configured to move two jaws. Those skilled in the art will realise that jaws may be arranged laterally i.e. in the side-by-side arrangement.

Where in the foregoing description, integers or elements are mentioned which have known, obvious or foreseeable equivalents, then such equivalents are herein incorporated as if individually set forth. Reference should be made to the claims for determining the true scope of the present invention, which should be construed so as to encompass any such equivalents. It will also be appreciated by the reader that integers or features of the invention that are described as preferable, advantageous, convenient or the like are optional and do not limit the scope of the independent claims. Moreover, it is to be understood that such optional integers or features, whilst of possible benefit in some embodiments of the invention, may not be desirable, and may therefore be absent, in other embodiments.

1. Forceps for use in ear, nose and throat procedures, the forceps comprising:
a forward handle and a rear handle;
at least one movable jaw proximate to, or at, a front of the forceps, said jaw being coupled to the rear handle such that movement of the rear handle causes movement of the at least one jaw between a first operating position and a second operating position,
the forward handle comprising a forward-facing digit contact surface and a rearward-facing digit contact surface.

2. Forceps according to claim 1 configured such that more than one jaw is coupled to the rear handle so that movement of the rear handle causes movement of said more than one jaws.

3. Forceps according to claim 1 configured such that one jaw is coupled to the rear handle such that movement of the rear handle causes movement of said one jaw, optionally wherein the movable jaw is an upper jaw or a lower jaw, and optionally an upper jaw.

4. Forceps according to claim 3 comprising a support which is rigidly coupled to the forward handle, the non-movable jaw being coupled to the support.

5. Forceps according to claim 4 comprising a jaw member comprising a jaw and an interface portion comprising a male or female portion for mating with a corresponding female or male portion provided by the support to secure the jaw member and therefore the jaw to the support.

6. Forceps according to claim 5 wherein said jaw interface portion is provided with a male part and the support is provided with a corresponding female part, and wherein the male part and the female part together form an interference-fit arrangement.

7. Forceps according to claim 1 comprising two jaws, wherein in the first operating position the at least one movable jaw is further away from the other jaw than when the at least one movable jaw is in the second operating position.

8. Forceps suitable for ear, nose and throat procedures, the forceps comprising:
a forward handle and a rear handle;
a movable first jaw proximate to, or at, a front of the device, said movable jaw being coupled to the forward or the rear handle such that movement of the respective forward or rear handle causes movement of the at least one jaw between a first operating position and a second operating position;
a jaw member comprising a second jaw which co-operates with the first jaw and an interface portion comprising a male or female portion which is mated with a respective female or male portion provided by a jaw support to form an interference fit to secure the jaw member to the jaw support.

9. Forceps according to claim 8 wherein the first jaw is coupled to the rear handle such that movement of the rear handle causes movement of the first jaw between a first operating position and a second operating position.

10. Forceps according to claim 8 wherein the movable jaw which is coupled to the respective handle is coupled to the handle with a movable coupling member.

11. Forceps according to claim 10 in which the coupling member is elongate and is pivotally attached to the respective handle, and is coupled to the respective handle such that movement of the respective handle causes linear translational motion of the coupling member.

12. Forceps according to claim 10 comprising a coupler to convert movement of the movable coupling member into movement of the movable jaw.

13. Forceps according to claim 10 wherein the movable jaw is mounted for rotation about a pivot, and the coupler comprises a pin and a groove arrangement, one of the pin and groove being associated with the movable jaw, the other of the pin and groove being associated with the movable coupling member.

14. Forceps according to claim 13 wherein the movable jaw is provided as a jaw member comprising a jaw portion and a jaw member coupler portion, the jaw member coupler portion comprising the pin or the groove of the pin and the groove arrangement.

15. Forceps according to claim 14 wherein the jaw member coupler portion comprises an aperture for facilitating pivoting movement of the jaw member.

16. Forceps according to claim 1 comprising a rearward facing digit contact surface and a forward facing digit contact surface, the rearward facing digit contact surface being below the forward facing digit contact surface, the forward-facing digit contact surface being concave when viewed from the front of the forceps, and the rearward-digit contact surface being concave when viewed from the rear of the forceps.

17. Forceps according to claim 8 wherein the first jaw is provided by a first jaw member comprising the first jaw and a jaw member coupler portion to facilitate movement of said jaw member.

18. A kit for assembly into forceps suitable for ear, nose and throat procedures, the kit comprising:
a jaw support; and
a plurality of sets of jaw members,
each set of jaw members comprising (i) a first jaw member comprising a first jaw and a jaw member coupler portion to facilitate movement of said jaw member when mounted for use and (ii) a second jaw member comprising a second jaw and a jaw interface portion comprising a male or female portion to facilitate mating with a corresponding female or male portion provided by the jaw support, thereby forming an interference fit to secure the second jaw member to the jaw support, the jaw member coupler portion of the first jaw member of each of the plurality of sets being substantially the same;
the jaw interface portion of the second jaw member of each of the plurality of sets being substantially the same; and
at least the first jaw or the second jaw of each of the plurality of sets being different.

19. A method of manufacturing forceps comprising:
providing a kit wherein the kit comprises:
a jaw support; and
a plurality of sets of jaw members,
each set of jaw members comprising (i) a first jaw member comprising a first jaw and a jaw member coupler portion to facilitate movement of said jaw member when mounted for use and (ii) a second jaw member comprising a second jaw and a jaw interface portion comprising a male or female portion to facilitate mating with a corresponding female or male portion provided by the jaw support, thereby forming an interference fit to secure the second jaw member to the jaw support,
the jaw member coupler portion of the first jaw member of each of the plurality of sets being substantially the same,
the jaw interface portion of the second jaw member of each of the plurality of sets being substantially the same, and
at least the first jaw or the second jaw of each of the plurality of sets being different;
selecting a set of jaw members; and
making forceps using the selected jaw members where the first jaw and the jaw member coupler portion facilitate movement of said jaw member.

20. The method according to claim 19 comprising packaging said forceps, sterilising said forceps, and delivering said forceps to a customer.
